(19) United States
(12) Patent Application Publication
(10) Pub. No.: US 2021/0033505 A1
(43) Pub. Date: Feb. 4, 2021

(54) METHODS OF MEMBRANE-BASED PROTEOMIC SAMPLE PREPARATION

(71) Applicant: CHILDREN'S MEDICAL CENTER CORPORATION, Boston, MA (US)

(72) Inventors: Hanno STEEN, Brighton, MA (US); Sebastian T. BERGER, Cambridge, MA (US); Saima AHMED, Woburn, MA (US)

(73) Assignee: CHILDREN'S MEDICAL CENTER CORPORATION, Boston, MA (US)

(21) Appl. No.: 17/067,296

(22) Filed: Oct. 9, 2020

Related U.S. Application Data
(63) Continuation of application No. 15/541,908, filed on Jul. 6, 2017.
(60) Provisional application No. 62/101,797, filed on Jan. 9, 2015.

(51) Int. Cl. G01N 1/40; G01N 33/68; B01L 3/00; B01D 61/14; B01D 65/02; B01D 69/02; B01D 71/34

(52) U.S. Cl. CPC G01N 1/4077; G01N 33/6803; B01L 3/50255; B01D 61/145; B01D 65/02; B01D 69/02; B01L 2300/0681; G01N 2001/4088; B01L 2300/165; B01L 2400/049; B01L 2400/0409; B01D 2321/16; B01D 2325/38; B01D 71/34

(57) ABSTRACT

A method for rapid isolation of a biological compound (e.g. protein) from an aqueous sample is described herein. The method uses a porous hydrophobic membrane that has an average pore size significantly greater than the size of the biological compound. The method permits the biological compound to attach to the membrane while the aqueous solvent rapidly moves through the membrane under the application of a vacuum. The biological compound that is attached to the membrane can be washed, optionally digested, and eluted for analysis.

### CROSS-REFERENCE TO RELATED APPLICATION

This application is a continuation application of Ser. No. 15/541,908 filed Jul. 6, 2017, which is a 35 U.S.C. § 371 National Phase Entry Application of International Application No. PCT/US2016/012591 filed Jan. 8, 2016, which designates the U.S. and claims benefit under 35 U.S.C. § 119(e) of U.S. Provisional Application No. 62/101,797, filed Jan. 9, 2015, the contents of which are incorporated herein by reference in their entireties.

### TECHNICAL FIELD

The present invention relates to proteomics and protein sample preparation.

### BACKGROUND

Mass spectrometry (MS)-based proteomics is moving increasingly into the translational and clinical research arena, where robust and efficient sample processing is progressively of particular importance. The conventional sample processing methods in proteomics, namely SDS-PAGE- or in-solution-based sample processing, are slow and laborious, and thus do not easily provide the reproducibility and throughput necessary to meet today's demands. A paradigm shift was the introduction of filter-aided sample processing method (FASP), which were initially described by Manza et al. (2005) [Manza, L. L., et al., Proteomics, 2005. 5(7): p. 1742-5; Liebler, D. C. and A. J. Ham, Nat Methods, 2009. 6(11): p. 785] and then fully realized in practice by Wisniewski et al. (2009) [Wisniewski, J. R., et al., Nat Methods, 2009. 6(5): p. 359-62]. These filter-aided methods make use of ultrafiltration membranes with molecular weight cut offs (MWCO) in the 10 to 30 kDa range to efficiently remove small molecules and salts, and to capture denatured proteins on a cellulose filter even if the molecular weight of the protein is much smaller than the nominal MWCO of the ultrafiltration membrane. Thus, the denaturation step is crucial to ensure that proteins much smaller than the nominal MWCO are efficiently retained by e.g. a 10 kDa MWCO filter.

In translational and clinical proteomics, which normally include large cohorts, the multi-titer plate is the preferred format for sample processing and storage. Although the application of FASP in the 96-well plate format has been described [Switzar, L., et al., Proteomics, 2013. 13(20): p. 2980-3; Yu, Y., et al., Anal Chem, 2014. 86(11): p. 5470-7], the major limitation of FASP in the 96-well plate is the much slower speed at which the 96-well plates have to be centrifuged: while a single ultrafiltration units withstands up to 14,000×g, the 96-well plate format can only be centrifuged at g-forces of up to ˜2,200×g. This significantly lower g-force for 96-well plates results in a slow liquid transfer, which in turn considerably prolongs the required centrifugation times to hours instead of tens of minutes for, in total, three to four centrifugation steps i) for the initial loading, reduction and alkylation, ii) for the different washing steps, and iii) for the elution [Switzar, L., et al., Proteomics, 2013. 13(20): p. 2980-3].

Independent of the format FASP is performed in, the conventional FASP also requires relative large volumes of high salt concentration for efficient elution of the tryptic peptides. Hence, reversed phase-based desalting of the samples is a prerequisite for subsequent LC/MS experiments. Apart from prolonging the entire FASP procedure, the numerous additional handling steps are potentially also associated with peptide losses [Naldrett, M. J., et al., J Biomol Tech, 2005. 16(4): p. 423-8].

Accordingly, there is an unmet need for fast sample processing methods for proteomics.

### SUMMARY

The technology described herein exploits the following two attributes of certain porous hydrophobic membranes for rapid isolation of a biological compound from an aqueous sample: (1) the biological compound can naturally attach to the membrane as a result of hydrophobic interactions; and (2) the membrane comprises pores of sufficient size for rapid liquid transfer across the membrane under the application of a vacuum. The biological compound can thus be isolated from the aqueous sample in any setup or device traditionally used for filtering, provided that the proper membrane is used. The technology described herein is particularly useful for the isolation of peptides or polypeptides from aqueous samples.

One aspect of the technology described herein relates to a method of separating a biological compound from an aqueous sample containing the biological compound, the method comprising: (i) introducing the aqueous sample to a well of a plate, wherein the well has a bottom comprising a porous hydrophobic membrane, and the sample is in contact with a first side of the porous hydrophobic membrane; (ii) applying a vacuum to a second side of the porous hydrophobic membrane, thereby drawing the aqueous sample through the porous hydrophobic membrane, wherein the biological compound associates with the porous hydrophobic membrane as aqueous solvent passes through; and (iii) introducing a solvent solution to the first side of the porous hydrophobic membrane to elute the biological compound from the porous hydrophobic membrane.

In one embodiment of the aspect noted above, the biological compound is a peptide or polypeptide.

In one embodiment, the method further comprises moving the hydrophobic membrane to a separate container after step (ii).

In one embodiment the method further comprises, after step (ii), a step of introducing a solution comprising a proteolytic enzyme to the first side of the porous hydrophobic membrane, thereby permitting the biological compound to be digested by the enzyme. In one embodiment, the proteolytic enzyme is trypsin. In one embodiment, the solution comprises an organic solvent. In one embodiment the organic solvent is acetonitrile or trifluoroethanol or combinations thereof.

In one embodiment, the solvent solution introduced in step (iii) comprises an organic solvent. In one embodiment, the organic solvent is acetonitrile or trifluoroethanol or combinations thereof.

In one embodiment, the average pore size of pores in the porous hydrophobic membrane is at least 50 nm in diameter. In one embodiment, the average pore size of pores in the porous hydrophobic membrane is in the range of 50 nm to 5 μm in diameter. In one embodiment, the average pore size of pores in the porous hydrophobic membrane is about 450 nm in diameter.

In one embodiment, the porous hydrophobic membrane is comprised of a hydrophobic polymer. In one embodiment, the hydrophobic polymer is selected from the group consisting of polyvinylidene difluoride (PVDF), polytetrafluoroethylene (PTFE), polyethylene, polysulfone, and polycarbonate.

In one embodiment, the solvent in the solvent solution introduced in step (iii) of the method is selected from the group consisting of acetonitrile, formic acid, methanol, ethanol, isopropanol, and combinations thereof.

In one embodiment, the method further comprises, after step (ii), repeating steps (i) and (ii) on the aqueous sample having passed through the porous hydrophobic membrane.

In one embodiment, the method further comprises washing the porous hydrophobic membrane prior to step (iii).

In one embodiment, the elution step (iii) comprises stepwise introduction and removal of solvent solution containing increasing concentrations of organic solvent. In one embodiment, the elution step (iii) comprises stepwise introduction and removal of a solution comprising 5%, 10%, 20% and 40% acetonitrile. In one embodiment, the elution step (iii) comprises stepwise introduction and removal of a solvent solution comprising 10%, 20% and 40% acetonitrile.

In one embodiment, the plate comprises a plurality of wells, and wherein the bottom of each well comprises a porous hydrophobic membrane. In one embodiment, the aqueous sample is introduced to the plurality of wells. In one embodiment, the plate is a 96-well plate.

In one embodiment, the aqueous sample is selected from the group consisting of a cell lysate, a tissue lysate, and a biofluid. In one embodiment, the biofluid is selected from the group consisting of urine, cerebrospinal fluid and blood, or more commonly blood fraction(s) such as serum or plasma.

In one embodiment, the aqueous sample is drawn through the porous hydrophobic membrane at a flow rate in the range of 50 uL/min to 1000 uL/min.

### BRIEF DESCRIPTION OF THE DRAWINGS

FIGS. 1A-1B show FASP vs. MStern Blot. FIG. 1A is a schematic showing comparison of the physical properties of the ultrafiltration membrane used for FASP and the membrane used for MStern Blot. FASP uses physical retention while in MStern blotting proteins are adsorbed onto the hydrophobic membrane surface. FIG. 1B is a plot showing time advantage of MStern blotting (blue curve) vs. FASP (yellow curve) without considering potentially different digestion times. Major time savers are the fast liquid transfer steps (1 min vs. 100 min; red) and the omission of any desalting (green).

FIGS. 2A-2C show performance comparison MStern Blot vs. FASP. FIG. 2A is a plot showing comparison of proteins identified from CSF (cerebrospinal fluid), HeLa lysate and urine after loading approx. 10 ug, 10 ug, and 15 ug, respectively. Each sample type was processed in quadruplicate. FIG. 2B is a plot showing comparison of the dynamic ranges of the identified proteins in three different biological samples (CSF, HeLa lysate and urine); MaxQuant-based iBAQ intensities are marked blue (MStern blotting) and yellow (FASP). FIG. 2C is a set of plots showing testing the loading capacity of the PVDF membrane used for MStern blotting based on proteins identified adsorbed to the PVDF membrane (i.e. MStern blotting, blue curve) and the respective flow through processed by FASP (red curve), in comparison to standard FASP of the same sample (yellow curve). A HeLa lysate was used. Values shown demonstrate average protein identifications.

FIGS. 3A-3C are a set of diagrams and plots showing comparison of the properties of the identified proteins. Venn diagram of the proteins and peptides identified from CSF (FIG. 3A), HeLa lysate (FIG. 3B) and urine (FIG. 3C). On the bottom, GO annotations (cellular compartment) of the method specific proteins, namely MStern blotting (blue) or FASP (yellow).

FIGS. 4A-4B are a set of plots showing investigation of physical & chemical properties for the sample type HeLa lysate. FIG. 4A is a set of plots showing comparison of three different properties: Molecular Weight (top), isoelectric pH (middle) and GRAVY score (bottom) on protein level. FIG. 4B is a set of plots showing investigation of chemical/physical property changes for: Molecular Weight (top), isoelectric pH (middle) and GRAVY score (bottom), on peptide level.

FIG. 5 is a set of plots showing correlation of FASP- and MStern Blotting-based protein quantifications based on the signal intensities of the intact peptide ions. Correlation of the Protein Pilot-derived signal intensities of the proteins identified in CSF, HeLa lysate and urine (see FIGS. 2A-2C): MStern blot vs. MStern blot (left), FASP vs. FASP (middle) and MStern blot vs. FASP (right).

FIGS. 6A-6B are a set of plots showing investigation of physical & chemical properties for the sample type CSF. FIG. 6A is a set of plots showing comparison of three different properties: Molecular Weight (top), isoelectric pH (middle) and GRAVY score (bottom) on protein level. FIG. 6B is a set of plots showing investigation of chemical/physical property changes for: Molecular Weight (top), isoelectric pH (middle) and GRAVY score (bottom), on peptide level.

FIGS. 7A-7B are a set of plots showing investigation of physical & chemical properties for the sample type urine. FIG. 7A is a set of plots showing comparison of three different properties: Molecular Weight (top), isoelectric pH (middle) and GRAVY score (bottom) on protein level. FIG. 7B is a set of plots showing investigation of chemical/physical property changes for: Molecular Weight (top), isoelectric pH (middle) and GRAVY score (bottom), on peptide level.

FIG. 8 is a set of plots showing correlation of FASP- and MStern Blotting-based protein quantifications based on spectral counts. Correlation of the proteins identified in CSF, HeLa lysate and urine (see FIGS. 2A-2C): MStern blot vs. MStern blot (left), FASP vs. FASP (middle) and MStern blot vs. FASP (right).

FIGS. 9A-9B are a set of plots showing fractionation of proteolytic peptides by differential elution with increasing amounts of acetonitrile. FIG. 9A shows the number of peptides identified in each fraction upon stepwise elution by stepwise increasing the amounts of acetonitrile from 0%, 5%, 10% to 40% and repeating the elution steps twice using the method described herein.

FIG. 9B shows the Venn diagram of number of unique and overlapping peptides identified in individual elution fractions obtained using stepwise elution with increasing amounts of 0%, 5%, 10% and 40% acetonitrile.

FIG. 10 is a bar diagram showing effect of presence of SDS in the aqueous sample containing the proteins. The bar diagram shows the number of proteins identified by the method described herein from cellular digests of Hela cells containing 2% SDS (1822) is comparable to that identified from Hela cells digests in absence of SDS (1849).

FIG. 11 shows optimization of digestion conditions for proteins contained in a cerebrospinal fluid sample in the presence of different concentrations of organic solvents, acetonitrile and/or trifluoroethanol. The figure shows the number of proteins identified using the method described herein in a cerebrospinal fluid sample digested in the presence of 0%, 5%, 10% or 15% acetonitrile or with 0%, 5% or 10% trifluoroethanol. The highest numbers of proteins were identified upon digestion in the presence of 0-10% acetonitrile and 0-5% trifluoroethanol.

### DETAILED DESCRIPTION

The technology described herein is based, in part, on the surprising discovery that porous membranes having pores significantly larger than proteins in size can be used to retain the proteins in a process akin to filtering. Another added advantage of large pores is rapid transfer of an aqueous solvent through the membrane.

The technology described herein is directed to a proteomic sample processing method that is compatible with multiwell plates and permits the simultaneous processing of multiple samples within a single workday. Specifically, the sample processing method described herein takes advantage of the efficient adsorption of proteins onto the surface of a porous hydrophobic membrane, even when the average size of the pores of the membrane is significantly larger than the size of the proteins. For example, the average pore size can be at least 10 times, at least 20 times, at least 30 times, at least 40 times, at least 50 times, at least 100 times, at least 200 times, at least 300 times, at least 400 times, at least 500 times, or at least 1000 times larger than the size of the proteins. Vacuum can be applied to hasten the speed of liquid transfer through the membrane. Once the proteins are attached on the membrane, they can be washed and eluted for further analysis (e.g., mass spectrometry such as electrospray ionization-based liquid chromatography/mass spectrometry (LC/MS)).

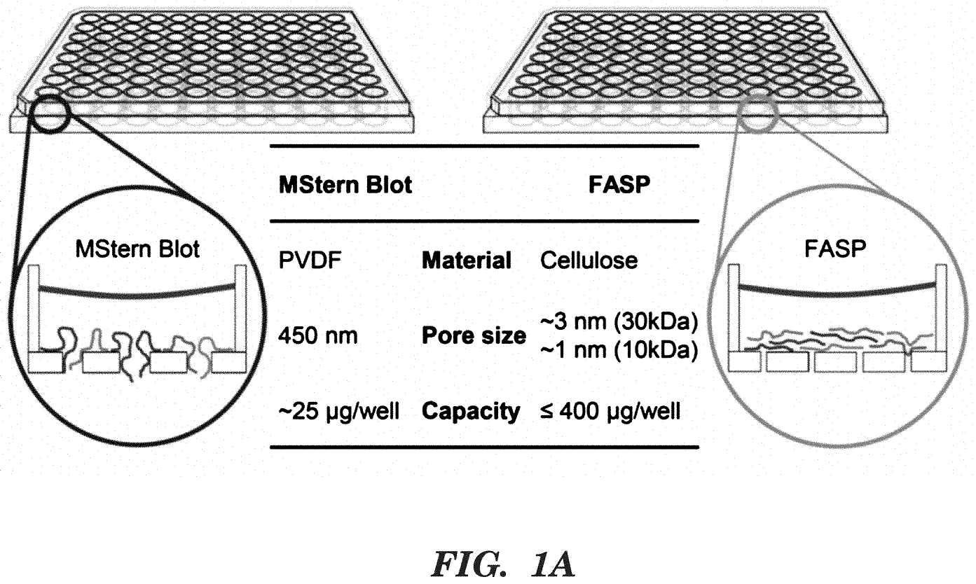

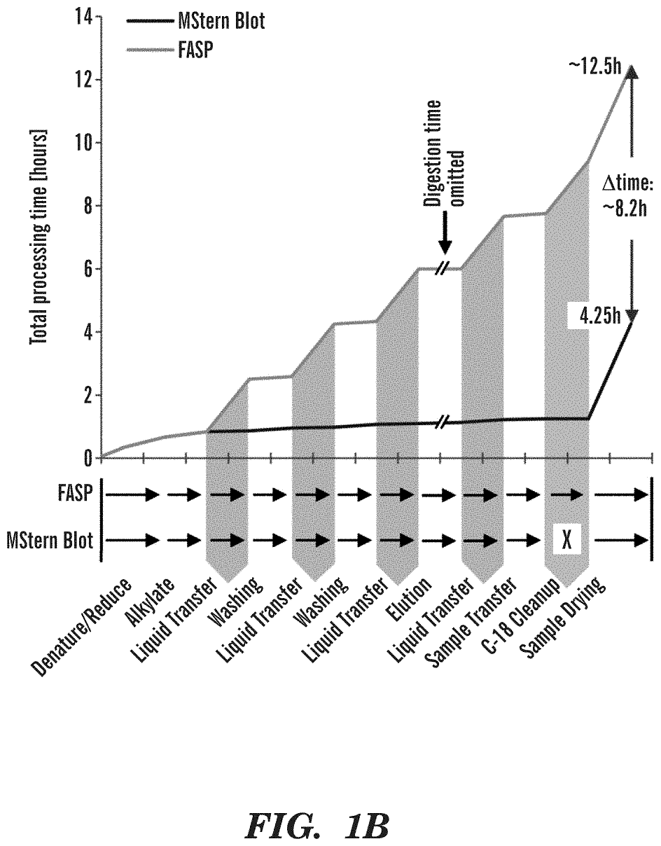

While the FASP method makes use of the size-based retention of proteins on top of a membrane, the method described herein uses porous membranes which have pores significantly larger than proteins in size. As shown in FIG. 1A, the ultrafiltration units that are used for the FASP method features a pore size of 1-3 nm (10-30 kDa MWCO) while the porous hydrophobic membrane used in the method described herein features pores of at least 10 times larger. These larger pores significantly reduce the force needed for efficient liquid transfer, thereby reducing the time requirement for the liquid transfer through the membrane, even when using low grade vacuum vs. centrifugation at tens of thousands times g-force.

One aspect of the technology described herein relates to a method of separating a biological compound from an aqueous sample containing the biological compound. The method comprises a step of introducing the aqueous sample to a well, the well having a bottom comprising a porous hydrophobic membrane. After the sample is introduced, it is in contact with a first side of the porous hydrophobic membrane. The method further comprises a step of applying a vacuum to a second side of the porous hydrophobic membrane. The application of vacuum draws the aqueous solvent of the sample through the porous hydrophobic membrane while the biological compound associates with the porous hydrophobic membrane. After the aqueous solvent passes through the membrane, it can be collected, e.g. in a container. The same filtering step can be repeated on the aqueous solvent once or more (e.g., twice, three times, four times, or more), which can increase the percentage of the biological compound associated with the membrane. Methods and systems (e.g., a pump) for generating a vacuum are known in the art. In one embodiment, the vacuum is less than 700 Torr, less than 600 Torr, less than 500 Torr, less than 400 Torr, less than 300 Torr, less than 200 Torr, less than 150 Torr, less than 100 Torr, less than 50 Torr, or less than 5 Torr. Generally, the stronger the vacuum, the faster the aqueous solvent is drawn through the porous hydrophobic membrane. The appropriate strength of the vacuum can be selected to balance the flow rate of liquid transfer and the time necessary for the biological compound to interact with the membrane. In one embodiment, the vacuum is in the range of 1.5 to 150 Torr. In one embodiment, the vacuum is in the range of 75 to 150 Torr. The flow rate can be in the range of 50 uL/min to 1000 uL/min. In one embodiment, the flow rate is in the range of 100 uL/min to 500 uL/min. In one embodiment, the flow rate is about 200 uL/min.

Additionally, the method further comprises a step of introducing a solvent to the first side of the porous hydrophobic membrane to elute the biological compound or fragment thereof from the porous hydrophobic membrane. As part of this elution step, vacuum can be applied again to facilitate liquid transfer.

It should be noted that while centrifugation can be used in place of vacuum in methods such as those described herein, the use of vacuum is simpler and does not require a centrifugation device.

After the biological compound is attached to the porous hydrophobic membrane, a solvent can be used to wash the biological compound, e.g. for removing salt and/or small molecules. In one embodiment, the solvent used for washing comprises ammonium bicarbonate.

In one embodiment, the biological compound is a peptide or polypeptide.

In one embodiment, the method further comprises, after the attachment of the biological compound to the porous hydrophobic membrane, a step of introducing a solution comprising one or more proteolytic enzymes to the first side of the porous hydrophobic membrane. The proteolytic enzymes can digest proteins bound to the membrane. Compositions and methods for digesting proteins (i.e., breaking down proteins into smaller peptide fragments) are known in the art. Examples of proteolytic enzymes include, but are not limited to, thermolysin, collagenase, trypsin, proteinase K, chymotrypsin, pepsin, pronase, endoproteinase Lys-C, Glu-C, Arg-C and papain. In one embodiment, the proteolytic enzyme is trypsin. The enzyme solution is generally contacted with the membrane and held under conditions (buffer, salt, temperature, time) that permit enzyme activity. Such conditions are known in the art for particular enzymes.

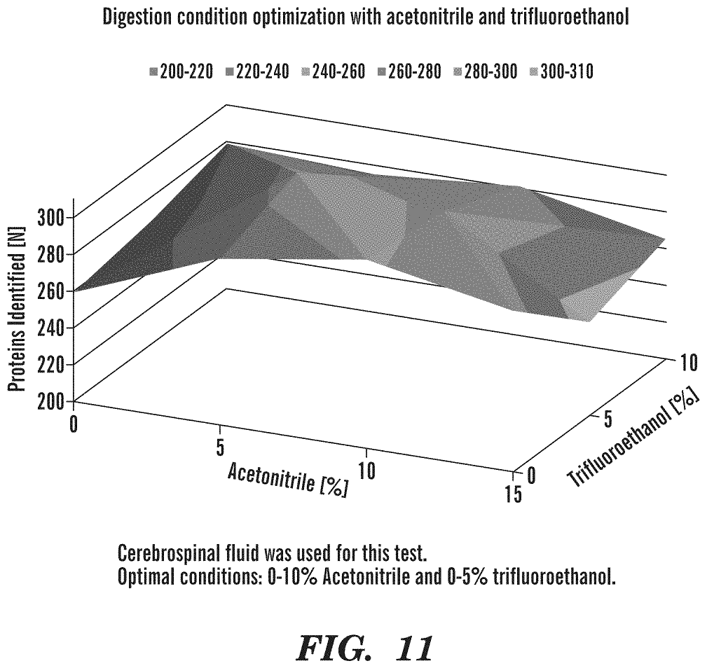

The methods described herein can tolerate the presence of solvents used in the preparation and/or processing of biological samples. In one aspect, as shown in FIG. 11, the solution used for digesting the biological sample attached to the porous membrane can comprise an organic solvent. Non-limiting examples of solvents include acetonitrile and trifluoroethanol.

A porous membrane can have through-holes or pore apertures extending vertically and/or laterally between two surfaces of the membrane, and/or a connected network of pores or void spaces (which can, for example, be openings, interstitial spaces or hollow conduits) throughout its volume. The porous nature of the membrane can be contributed by an inherent physical property of the selected membrane material, and/or introduction of conduits, apertures and/or holes into the membrane material.

The pores of the membrane (including pore apertures extending through the membrane from the top to bottom surfaces thereof and/or a connected network of void space within the membrane) can have a cross-section of any shape. For example, the pores can have a pentagonal, circular, hexagonal, square, elliptical, oval, diamond, and/or triangular shape. The pore shape can also be irregular.

The average pore size of pores in the porous hydrophobic membrane can have any dimension provided that it permits the aqueous solvent to pass through the membrane within a reasonable amount of time and the biological compound to attach to the surface (either exterior or interior) of the membrane. In one embodiment, the average pore size of pores in the porous hydrophobic membrane is at least 50 nm in diameter. In one embodiment, the average pore size of pores in the porous hydrophobic membrane is at least 100 nm in diameter. In one embodiment, the average pore size of pores in the porous hydrophobic membrane is at least 150 nm in diameter. In one embodiment, the average pore size of pores in the porous hydrophobic membrane is at least 200 nm in diameter. In one embodiment, the average pore size of pores in the porous hydrophobic membrane is at least 250 nm in diameter. In one embodiment, the average pore size of pores in the porous hydrophobic membrane is at least 300 nm in diameter. In one embodiment, the average pore size of pores in the porous hydrophobic membrane is at least 350 nm in diameter. In one embodiment, the average pore size of pores in the porous hydrophobic membrane is at least 400 nm in diameter.

In one embodiment, the average pore size of pores in the porous hydrophobic membrane is in the range of 50 nm to 5 μm in diameter, 100 nm to 5 μm in diameter, 150 nm to 5 μm in diameter, 200 nm to 5 μm in diameter, 250 nm to 5 μm in diameter, 300 nm to 5 μm in diameter, 400 nm to 5 μm in diameter, 400 nm to 4 μm in diameter, 400 nm to 3 μm in diameter, 400 nm to 2 μm in diameter, or 400 nm to 1 μm in diameter.

In one embodiment, the average pore size of pores in the porous hydrophobic membrane is 450 nm in diameter.

In one embodiment, the pore apertures can be randomly or uniformly distributed (e.g., in an array or in a specific pattern, or in a gradient of pore sizes) on the membrane. The spacing between the pore apertures can vary.

In one embodiment, the surface area of the membrane can be configured to provide a sufficient area for the biological compound to attach to.

The membrane can have any thickness provided that the selected thickness permits the membrane to maintain its physical integrity during the application of a vacuum. The thickness of the membrane should also permit the aqueous solvent to pass through the membrane within a reasonable amount of time.

In one embodiment, the porous hydrophobic membrane is comprised of a hydrophobic polymer. Any hydrophobic polymer can be applicable in the technology described herein. Non-limiting examples of hydrophobic polymers include polyvinylidene difluoride (PVDF), polytetrafluoroethylene (PTFE), polyethylene, polysulfone, and polycarbonate. In one embodiment, the porous hydrophobic membrane is made of PVDF. Porous hydrophobic membranes are commercially available from vendors such as VWR.

The well where the aqueous sample is introduced can be a part of a plate. In one embodiment, the plate comprises a plurality of wells (e.g. 2, 3, 4, 5, 6, 7, 8, 9, 10, or more), and the bottom of each well comprises a porous hydrophobic membrane. In one embodiment, the plate is a multiwell plate such as a 6-, 12-, 24-, 48-, 96-, 384-, or 1536-well plate. Multiwell plates comprising hydrophobic membranes on the well bottoms are commercially available for filtration applications from vendors such as Pall Co., Sigma Aldrich, Millipore, and VWR.

The well where the aqueous sample is introduced can also be a funnel.

In another aspect, the well where the aqueous sample is introduced can be a part of a stackable assembly comprising of more than one stacked well. The aqueous sample can be introduced in the top well which can be a part of a plate, the bottom of which comprises a porous hydrophobic membrane. The top plate can be stacked onto a second collection well, wherein the eluted biological sample or peptides are collected, which can be a part of second plate. The stackable assembly can be attached to a vacuum source. The plate can be a multiwell plate such as a 6-, 12-, 24-, 48-, 96-, 384-, or 1536-well plate. Such multiwell plate assemblies are commercially available from vendors such as Millipore and are described for example in U.S. Pat. No. 7,588,728 B2.

In one embodiment, the membrane can be excised and transferred to a second container after introduction of the aqueous sample and attachment or association of the biological sample onto the porous membrane. The subsequent sample processing and elution can be carried out in the second container.

A variety of solvents can be used to elute the biological compound or fragment thereof. For example, the solvent can be acetonitrile, formic acid, methanol, ethanol, isopropanol, or combinations thereof. The resulting solution comprising the biological compound or fragment thereof can be subjected to drying and/or analysis.

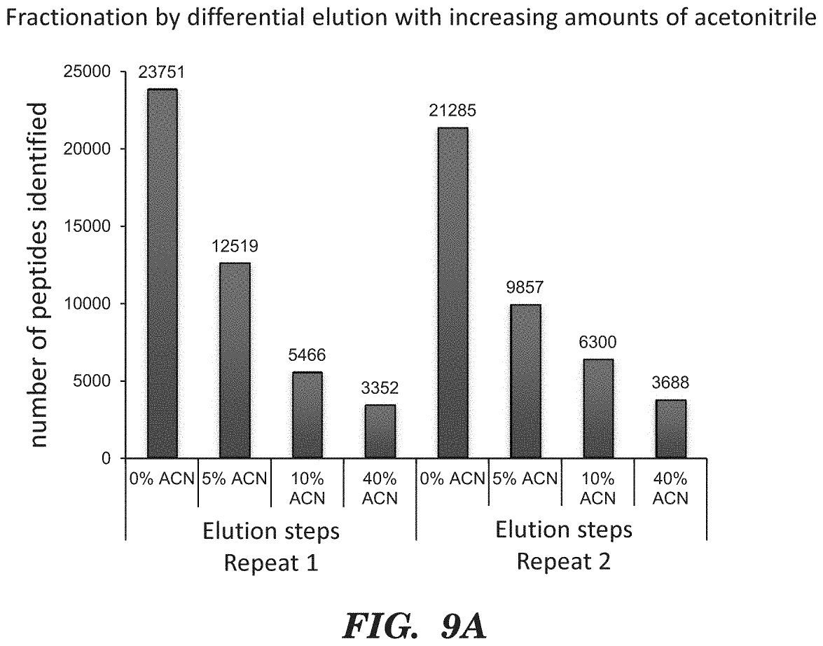

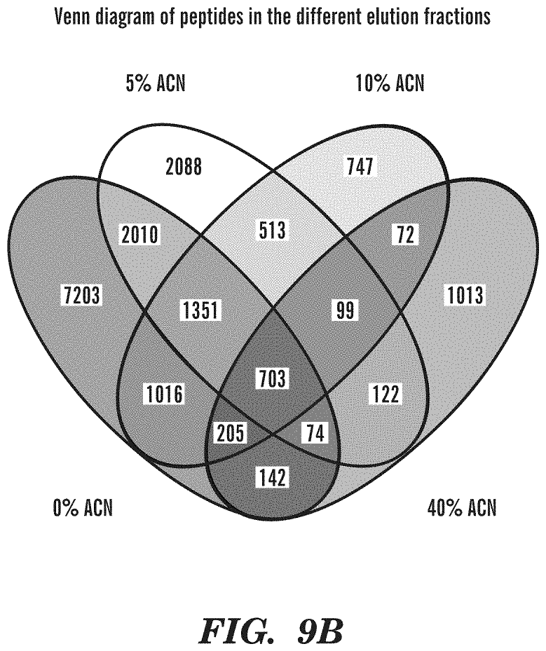

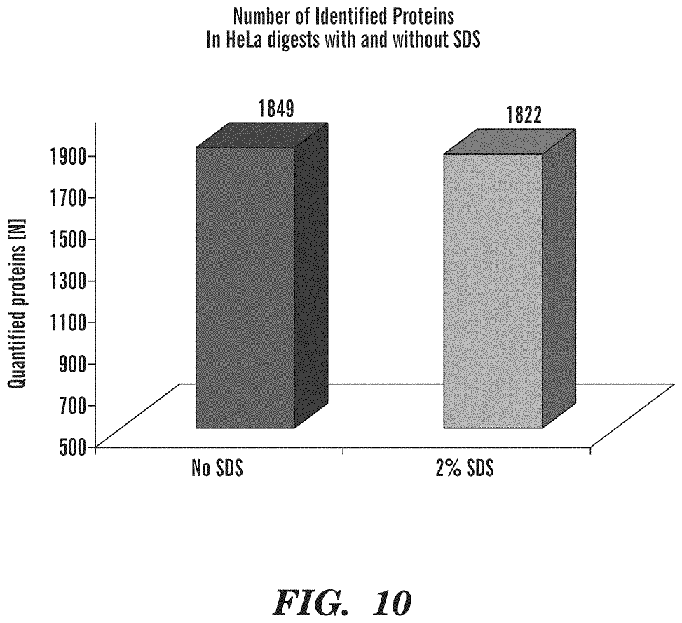

In one embodiment, the biological compound or fragment thereof can be eluted using a stepwise or fractional elution procedure. The procedure involves changing the composition of the solvent used to elute in a stepwise manner. The composition can be changed for example, by increasing the amounts of organic solvents in successive elutions. Under these conditions, the peptides elute according to their hydrophobicity, such that the procedure results in fractionation of peptides with increasing hydrophobicity in different elution fractions. A non-limiting example of stepwise elution or fractionation includes, digestion of biological sample with solvent comprising 0% acetonitrile and then successive elution with solvent containing 5%, 10%, 20% and 40% acetonitrile. Another non-limiting example is digestion of biological sample with solvent containing at least 5% acetonitrile and then successive elution with solvent comprising 10%, 20% and 40% acetonitrile. The stepwise elution with increasing amounts of organic solvents can be carried out more than once as shown for example in FIG. 9. While most often applicable to the elution of peptides following digestion, it is also contemplated that the stepwise elution approach can be used to fractionate undigested proteins in a sample bound to a membrane to effect a crude separation on the basis of hydrophobicity.

The aqueous sample comprising the biological compound can be a sample taken or isolated from a biological organism. Exemplary aqueous samples include, but are not limited to, a biofluid sample (e.g. a cerebrospinal fluid, blood, serum, plasma, urine, or saliva) a cell lysate, a tissue lysate, and combinations thereof. The aqueous sample can be obtained by removing a sample from a subject, but can also be accomplished by using previously sample (e.g. isolated at a prior time point and isolated by the same or another person). In addition, the aqueous sample can be a freshly collected or a previously collected sample.

In some embodiments, the aqueous sample can be an untreated aqueous sample. As used herein, the phrase “untreated aqueous sample” refers to an aqueous sample that has not had any prior sample pre-treatment except for dilution and/or suspension in a solution. Exemplary methods for treating an aqueous sample include, but are not limited to, centrifugation, filtration, sonication, homogenization, heating, freezing and thawing, and combinations thereof. In some embodiments, the aqueous sample can be thawed before employing the methods described herein. In some embodiments, the aqueous sample is a clarified sample, for example, by centrifugation and collection of a supernatant comprising the clarified sample.

In some embodiments, the aqueous sample can be a pre-processed sample, for example, supernatant or filtrate resulting from a treatment selected from the group consisting of centrifugation, filtration, thawing, purification, extraction, and any combinations thereof. The methods described herein can tolerate the presence of reagents commonly used in the preparation or processing of biological samples. For example, in some embodiments, the aqueous sample can be treated with or contain a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. Another exemplary reagent is Tris(hydroxymethyl)aminomethane (TRIS), which is generally a component of buffer solution. Other agents can be used to effect the separation of proteins and other biological molecules from materials with which they are associated, e.g., in a tissue or cell. Such reagents can be employed for solubilization of biological molecules, denaturation of biological molecules, and/or for reduction and alkylation of disulfide bonds of proteins. Non-limiting examples of denaturing reagents include detergents such as sodium dodecyl sulphate (SDS), sodium lauroyl sarcosinate (Sarkosyl), Polysorbate 20 (Tween 20), urea and guanidinium chloride. In one embodiment, the aqueous sample can contain as much as 2% SDS. Non-limiting examples of reducing reagents include dithiotreitol (DTT) and β-mercaptoethanol. Non-limiting examples of alkylating reagents include iodoacetamid (IAA). Where the presence of each of these types of reagents commonly used in the preparation of biological samples is tolerated by the methods described herein, the methods provide a robust alternative to existing methods that require that such agents either not be used, or that require time-consuming and/or yield-reducing steps to remove them. One of skill in the art can determine the impact of other agents upon the relative binding and/or elution of peptides from the porous membrane as described herein.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are described herein.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein, the term “porous” generally refers to a material that is permeable. The term “permeable” as used herein means a material that permits passage of a fluid and/or a molecule. The permeability of the membrane to individual materials of interest/species can be determined based on a number of factors, including, e.g., material property of the membrane (e.g., pore size, and/or porosity), interaction and/or affinity between the membrane material and individual species/materials of interest, individual species size, concentration gradient of individual species between both sides of the membrane, elasticity of individual species, and/or any combinations thereof.

As used herein, the term “hydrophobic” refers to a characteristic of a material that is water-repellent. A surface comprising a hydrophobic material can have a contact angle of water of 90° or greater.

As used herein, the term “biological compound” refers to a compound or molecule that is of biological origin.

As used herein, the terms “protein” and “polypeptide” are used interchangeably to designate a series of amino acid residues connected to each other by peptide bonds between the alpha-amino and carboxy groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “introduce” in the context of a solvent or sample means placing the solvent or sample into a well or onto a membrane.

The term “statistically significant” or “significantly” refers to statistical significance and generally means a two standard deviation (2SD) or greater difference.

As used herein, the term “much smaller than the nominal MWCO” refers to a size that is 70% of the nominal MWCO or less, 60% of the nominal MWCO or less, 50% of the nominal MWCO or less, 40% of the nominal MWCO or less, 30% of the nominal MWCO or less, 20% of the nominal MWCO or less, 10% of the nominal MWCO or less, 1% of the nominal MWCO or less.

Other than in the operating examples, or where otherwise indicated, all numbers expressing quantities of ingredients or reaction conditions used herein should be understood as modified in all instances by the term “about.” The term “about” when used in connection with percentages may mean±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Embodiments of various aspects described herein can be defined in any of the following numbered paragraphs:
1. A method of separating a biological compound from an aqueous sample containing the biological compound, the method comprising: (i) introducing the aqueous sample to a well of a plate, wherein the well has a bottom comprising a porous hydrophobic membrane, and the sample is in contact with a first side of the porous hydrophobic membrane; (ii) applying a vacuum to a second side of the porous hydrophobic membrane, thereby drawing the aqueous sample through the porous hydrophobic membrane, wherein the biological compound associates with the porous hydrophobic membrane as aqueous solvent passes through; and (iii) introducing a solvent solution to the first side of the porous hydrophobic membrane to elute the biological compound from the porous hydrophobic membrane.
2. The method of paragraph 1, wherein the biological compound is a peptide or polypeptide.
3. The method of paragraph 1, further comprising moving the hydrophobic membrane to a separate container after step (ii).
4. The method of any of paragraphs 1-3, further comprising, after step (ii), a step of introducing a solution comprising a proteolytic enzyme to the first side of the porous hydrophobic membrane, thereby permitting the biological compound to be digested by the enzyme.
5. The method of paragraph 4, wherein the proteolytic enzyme is trypsin.
6. The method of paragraph 4, wherein the solution introduced after step (ii) comprises an organic solvent.
7. The method of paragraph 6, wherein the organic solvent is acetonitrile, trifluoroethanol, a combination thereof.
8. The method of any of paragraphs 1-7, wherein the solvent solution introduced in step (iii) comprises an organic solvent.
9. The method of paragraph 8, wherein the organic solvent is acetonitrile, trifluoroethanol, a combination thereof.
10. The method of any one of paragraphs 1-9, in which the average pore size of pores in the porous hydrophobic membrane is at least 50 nm in diameter.
11. The method of any one of paragraphs 1-10, in which the average pore size of pores in the porous hydrophobic membrane is in the range of 50 nm to 5 μm in diameter.
12. The method of any one of paragraphs 1-11, in which the average pore size of pores in the porous hydrophobic membrane is about 450 nm in diameter.
13. The method of any one of paragraphs 1-12, wherein the porous hydrophobic membrane is comprised of a hydrophobic polymer.
14. The method of paragraph 13, wherein the hydrophobic polymer is selected from the group consisting of polyvinylidene difluoride (PVDF), polytetrafluoroethylene (PTFE), polyethylene, polysulfone, and polycarbonate.
15. The method of any one of paragraphs 1-13, wherein the solvent in the solvent solution introduced in step (iii) is selected from the group consisting of acetonitrile, formic acid, methanol, ethanol, isopropanol, and combinations thereof.
16. The method of any one of paragraphs 1-15, further comprising, after step (ii) repeating steps (i) and (ii) on the aqueous sample having passed through the porous hydrophobic membrane.
17. The method of any one of paragraphs 1-16, further comprising washing the porous hydrophobic membrane prior to step (iii).
18. The method of paragraph 8 or paragraph 9, wherein elution step (iii) comprises stepwise introduction and removal of solvent solution containing increasing concentrations of organic solvent.
19. The method of paragraph 18, wherein the elution step (iii) comprises stepwise introduction and removal of a solvent solution comprising 5%, 10%, 20% and 40% acetonitrile.
20. The method paragraph 18, wherein the elution step (iii) comprises stepwise introduction and removal of a solvent solution comprising 10%, 20% and 40% acetonitrile.
21. The method of any one of paragraphs 1-20, wherein the plate comprises a plurality of wells, and wherein the bottom of each well comprises a porous hydrophobic membrane.
22. The method of paragraph 21, wherein the aqueous sample is introduced to the plurality of wells.
23. The method of paragraph 21 or paragraph 22, wherein the plate is a 96-well plate
24. The method of any one of paragraphs 1-23, wherein the aqueous sample is selected from the group consisting of a cell lysate, a tissue lysate, and a biofluid.
25. The method of paragraph 24, wherein the biofluid is selected from the group consisting of urine, blood, serum, cerebrospinal fluid, and plasma.
26. The method of any one of paragraphs 1-25, wherein the aqueous sample is drawn through the porous hydrophobic membrane at a flow rate in the range of 50 uL/min to 1000 uL/min.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example 1: MStern Blotting—Ultrafast PVDF Membrane-Based Proteomic Sample Preparation

A 96-well plate compatible proteomic sample processing approach that allows the preparation of 96 samples or multiples thereof within a single workday is described herein. The larger pore size used in the approach described herein results in a very fast liquid transfer through the membrane, thereby significantly reducing the processing time. This approach is carried out on different clinical samples with varying complexity (urine and cerebrospinal fluid) as well as on highly complex cell culture samples (HeLa lysate). Equal or even higher numbers of proteins were identified with this new approach compared to FASP. Surprisingly, protein quantification is not compromised. Since vacuum manifolds are sufficient for the sample transfer and residual salts occur only in low concentrations, samples are fully compatible with direct injections into LC/MS systems without prior offline desalting. Thus, this new sample processing method, named “MStern blotting” herein, allows for easy automation and truly high throughput sample processing.

### Materials and Methods

Cell Culture. Human cervical cancer cells (HeLa) were propagated in Dulbecco's modified Eagle's medium (DMEM; 11965; Life technologies). Upon achieving 85-90% confluency, the growth media was aspirated and the cells were washed three times with 5 ml ice-cold PBS. One ml of modified RIPA buffer (150 mM NaCl, 50 mM Tris/HCl pH 7.4, 1% NP-40, 0.1% Sodium Deoxycholate, 1 mM EDTA) supplemented with 1× Roche Complete protease inhibitors, was add to each plate of cells and incubated for 30 min on ice. Cells were scraped with a cell scraper, collected in Eppendorf tubes and vortexed for 1 min. Cellular debris and other particulate matter was pelleted by centrifugation at 20,000×g at 4° C.; the supernatant was recovered for further use.

Protein Concentration Determination. Protein concentration was determined by using the Bradford Assay [7] (Bio-Rad DC™ Protein Assay) following the manufacturer's protocol. The standard curve was established using a stock solution of 20 mg/ml bovine serum albumin (BSA) and final concentrations of 0.25 mg/ml, 0.5 mg/ml, 1 mg/ml, 1.5 mg/ml and 2.0 mg/ml. After incubation at room temperature (RT) the final measurement was performed in a microplate spectrophotometer (Bio-Rad Model 680) at a wavelength of 595 nm.

MStern Blot. Undiluted neat urine (150 μl, i.e. ˜15 μg of protein) was added to a mixture of 150 μg urea and 30 μl dithiothreitol (DTT) (100 mM in 1M Tris/HCl pH 8.5). Diluted Hela cell lysates (10 μg in 100 μl 50 mM ammonium bicarbonate (ABC)) or neat CSF (10 μl, i.e. ˜10 μg of protein) was added to 100 μg urea and 20 μl DTT. The resulting solution was incubated for 20 min at 27° C. and 1100 rpm in a thermo mixer. Reduced cysteine side chains were alkylated with 50 mM iodoacetamide (IAA; final concentration) and incubation for 20 min in the dark at 27° C. and 750 rpm.

The hydrophobic PVDF membrane in a 96-well plate format (MSIPS4510, Millipore) was pre-wetted with 150 μl of 70% ethanol and equilibrated with 300 μl urea supernatant (˜8.3M urea). These and all subsequent liquid transfers were carried out using a fitted 96-well microplate vacuum manifold (MAVM0960R, Millipore)

Each sample was drawn three times through the PVDF membrane, although later experiments have shown that a single loading step is sufficient. The addition of Ca²⁺ was also tested, which had been described as beneficial for the protein binding onto PVDF membranes.

After protein adsorption of the proteins onto the membrane, it was washed twice with 50 mM ABC. Protein digestion was performed with sequencing grade trypsin (V5111, Promega) at a nominal enzyme to substrate ratio of 1:15. To this end, 100 μl digestion buffer (5% acetonitrile (ACN; v/v), 50 mM ABC and trypsin) were added to each well. Reducing the digestion buffer down to 50 μl does not affect the digestion performance.

After incubation for 2 hours at 37° C. in a humidified incubator, the remaining digestion buffer was evacuated. Resulting peptides were eluted twice with 150 μl of 40% ACN (v/v)/0.1% (v/v) formic acid (FA) each. Upon pooling, the peptide solutions were dried in a vacuum concentrator. Lyophilized samples were stored at −20° C. until further analysis.

Filter assisted sample preparation (FASP). The filter assisted sample preparation method was carried out as previously described [3]. In short: Proteins were first denatured and reduced by adding 100 μl sample to 100 μg urea supplemented with 20 μl DTT. For the different sample types, namely urine, CSF and HeLa lysate, a nominal protein content of 15 μg, 10 μg and 10 μg, respectively was used for analysis. After alkylation of reduced cysteine side chains with 50 mM IAA (final concentration), denatured proteins were captured on a 10 kDa MWCO spin filter (MRCPRT010, Millipore) and washed twice with 50 mM ABC. Protein digestion was performed with sequencing grade trypsin (V5111, Promega) at a nominal enzyme to substrate ratio of 1:50. After incubation over night with 100 μl digestion buffer (trypsin in 50 mM ABC), resulting peptides were eluted with 30 μl 0.5M sodium chloride (NaCl).

Peptide elutes were desalted with reversed phase-based TARGA C-18 spin tips (SEMSS18R, Nest Group) prior to LC-MS/MS analysis. Lyophilized samples were stored at −20° C. until further analysis.

LC-MS/MS Analysis. Peptides were reconstituted in loading buffer (5% ACN (v/v), 5% FA (v/v)). LC-MS/MS analysis was performed on a microfluidic chip system (EK425, Eksigent) coupled to a TripleToF 5600+(AB Sciex) mass spectrometer. Tryptic digests (˜1 μg) were loaded onto a trap column (ReproSil-Pur C₁₈-AQ, 200 μm×0.5 mm, 3, 3 μm) and subsequently separated on a ReproSil-Pur C₁₈-AQ analytical column chip (75 μm×15 cm, 3 μm) at a flow rate of 300 nl/min. A linear gradient from 95% to 65% buffer A (0.2% formic acid in HPLC water; buffer B: 0.2% formic acid in acetonitrile) within 60 min was applied. Samples were ionized applying 2.3 kV to the spray emitter. Analysis was carried out in a data-dependent mode. Survey MS1 scans were acquired for 200 msec. The quadrupole resolution was set to ‘UNIT’ for MS2 experiments which were acquired for 50 msec in a ‘high intensity’ mode. Following switch criteria were used: charge: 2+ to 4+; minimum intensity: 100 counts per second (cps). Up to 35 ions were selected for fragmentation after each survey scan. Dynamic exclusion was set to 17 s.

Data Analysis. Acquired MS raw files (WIFF) were analyzed using ProteinPilot (version 4.5.1; AB Sciex) using the human UniProtKB database (Homo sapiens, ˜68,000 sequences, version 06-2014). The ‘thorough’ search mode was used. Of note: ProteinPilot does not require the definition of an allowable number of missed cleavages or mass tolerances. Commonly occurring laboratory contamination protein sequences (cRAP, version 2012.01.01) were added to the UniProt database.

For the label free quantification, either peptide-spectrum matches were counted for spectral counting-based quantification [8] or by extracting the precursor intensities from the spectral summaries generated by ProteinPilot. Intensity-based absolute protein quantitation (iBAQ) [9] for dynamic range analysis, MaxQuant [10] (version 1.5.1) was used. Briefly, the acquired WIFF files were loaded into MaxQuant and searched against the human UniProtKB database (Homo sapiens, ˜68,000 sequences, version 06-2014). For quantification, the ‘iBAQ’ and ‘label-free quantification’ (LFQ) were selected. Default settings were used for the analyses.

Gene Ontology (GO) annotations were established by FunRich (http://www.funrich.org). Venn diagrams were generated using the online available tool Venny (http://bioinfogp.cnb.csic.es/tools/venny/). For the calculation of chemical and physical properties, online tools such as ExPASy (http://www.expasy.org) and GRAVY Calculator (http://www.gravy-calculator.de) were used.

### Results and Discussion

FASP Vs. MStern Blot

Filter-based sample processing in general and FASP in particular have replaced SDS-PAGE-based processing methods as the gold standard for generic sample processing in proteomics due to their sensitivity, wide applicability, and robustness. Despite a multitude of advantages, FASP or FASP-like methods have the drawback of not being readily compatible with 96 well plate formats because of the small pore size of the cellulose-based ultrafiltration membranes, which requires very long centrifugation times when used in the 96-well plate format. Cellulose ultrafiltration membranes that are used for the FASP approach feature a pore size of 1-3 nm (10 to 30 kDa MWCO) whilst the hydrophobic PVDF membranes used for sterilization filtration feature pores in the size range of 220 to 450 nm (see FIG. 1A). These 100 times larger pores overcome the major drawback of conventional FASP by significantly reducing the force needed for efficient liquid transfer, thereby reducing the time requirements for the liquid transfer through the membrane by up to 2 orders of magnitude even when using low grade (e.g. house) vacuum vs. centrifugation at tens of thousands times g-force. While the FASP approach makes use of the size-based retention on top of the membrane, the MStern blot approach makes use of the efficient adsorption of proteins onto the large hydrophobic surface of the PVDF membrane. Due to the different mode of retention, i.e. adsorption instead of size-based retention as in the case of FASP, the capacity of PVDF-based protein processing is theoretically in the 25 μg/well range (100 μg/cm²). This is lower than in conventional FASP, but still plentiful given the sensitivities of current LC/MS systems where rarely more than 1 μg is injected per analysis run.

FASP in individual ultrafiltration units is an easy and efficient way of processing samples because these units withstand centrifugal forces of up to 14,000×g, which ensures rapid liquid transfers. However, large-scale implementation using 96-well plates requires swinging-bucket rotors for centrifugation-based liquid transfer, and this type of rotors caps the centrifugal forces at ˜2,200×g, such that individual liquid transfer steps take 1 to 2 hours [4, 5]. In contrast to the ultrafiltration membranes, the use of large pore PVDF membranes for the protein sample processing enables very fast and easy liquid transfer with a vacuum manifold connected to low-grade house vacuum. The liquid transfer with this set-up can be as fast as 10 seconds if a small number of samples are processed on a plate or up to 2 minutes if all positions of the 96-well plate are in use. This significantly accelerated liquid transfer results in major time savings for the MStern blotting sample processing in comparison to FASP (see FIG. 1B).

Besides a faster liquid transfer through the membrane, further time savings are realized by the post-digestion peptide elution, which uses a simple mixture of acetonitrile and formic acid instead of concentrated salt solutions as in the case of FASP. The residual amount of ammonium bicarbonate salts are further reduced by the subsequent vacuum centrifugation, such that the samples are ready for LC/MS analysis once they have been evaporated to dryness. In contrast, FASP requires a lengthy and expensive reversed phase-based desalting of the digests. Together with the faster liquid transfers, all time savings add up to more than 8 hours when processing samples with MStern blotting instead of FASP. In addition, the use of vacuum manifolds also allows for easier automation when compared to FASP which requires centrifugation.

Performance of MStern Blot

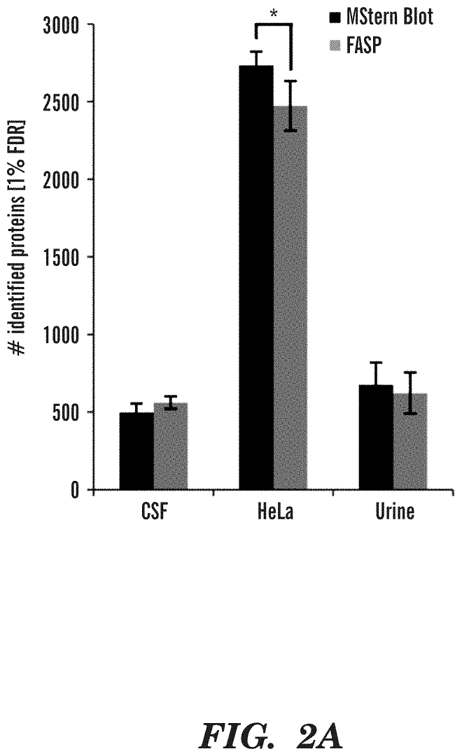

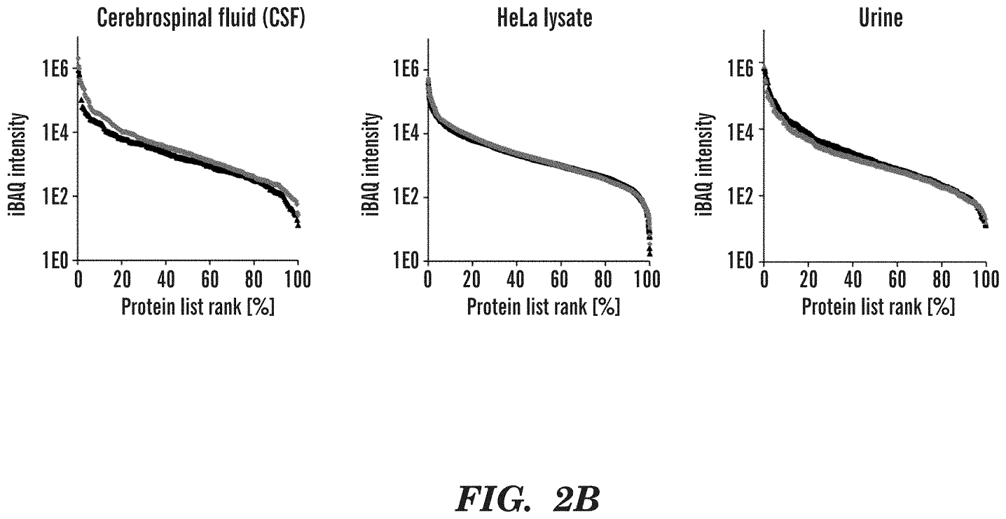

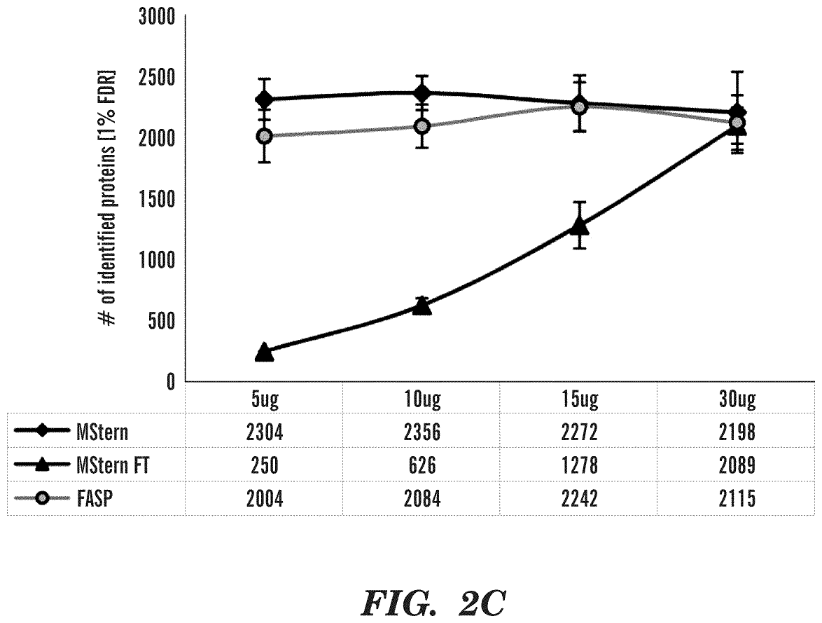

After establishing that using hydrophobic PVDF instead of hydrophilic regenerated cellulose as in the case of FASP allows for significant time savings, the compatibility of adsorption of complex protein mixtures with tryptic digestion was investigated. The digestion of individual proteins adsorbed onto PVDF membranes had been described before [11-13]. However, it was not evident that similar approaches would also work for highly complex protein mixtures quickly loaded by drawing dilute protein solutions through the membrane instead of e.g. slow electroblotting [11]. Thus, to test whether adsorption to hydrophobic PVDF is compatible with proteomic studies on complex protein mixtures, three different types of samples were used: neat urine, neat cerebrospinal fluid (CSF), and a highly complex whole cell (HeLa) lysate (see FIG. 2A).

The initial digestion optimization resulted in conditions which match or exceed the performance of FASP; thus, a more thorough optimization should provide even better results. Four aliquots for each sample type were processed and tryptic digests of the different sample types were analyzed by LC-MS/MS using a 1-hour gradient. These analyses identify 497±58, 2733±160, and 676±143 proteins from neat CSF, HeLa lysate and neat urine, respectively (FIG. 2A). The FASP-based processing of 4 aliquots of the same samples resulted in 561±40, 2473±89, and 622±133 proteins for neat CSF, HeLa lysate and neat urine, respectively. Also the dynamic ranges of the identified proteins as determined using the iBAQ method [9], was similar for both sample processing methods: ˜5 orders of magnitude of the two neat body fluids and 6 orders of magnitude for the HeLa cell lysate (FIG. 2B). These numbers clearly showed that the MStern blotting approach gives protein identification rates at least as good as FASP, irrespective of the nature and complexity of the sample.

Next, the loading capacity of the PVDF membrane was tested. To this end, 5, 10, 15 and 30 μg of HeLa lysate were loaded into individual wells of the PVDF membrane-equipped 96-well plate. The flow throughs of the loading and washing solutions were collected and subsequently processed using FASP. In parallel, identical amounts of protein were directly processed with FASP. The results are shown in FIG. 2C. In summary, FASP and MStern blotting resulted in similar number of identified proteins (while MStern consistently identified more than FASP as already shown in FIG. 2A), irrespective of the amount of protein processed. In contrast, the number of proteins identified in the flow through of the MStern blot-based processing steadily increases such that at a nominal loading of 30 μg, as many proteins are identified in the flow-through as in adsorbed fraction. Based on these numbers, not more than ˜10 μg of protein should be loaded into each well.

Detecting Biases in Proteins Identified in Method-Specific Samples

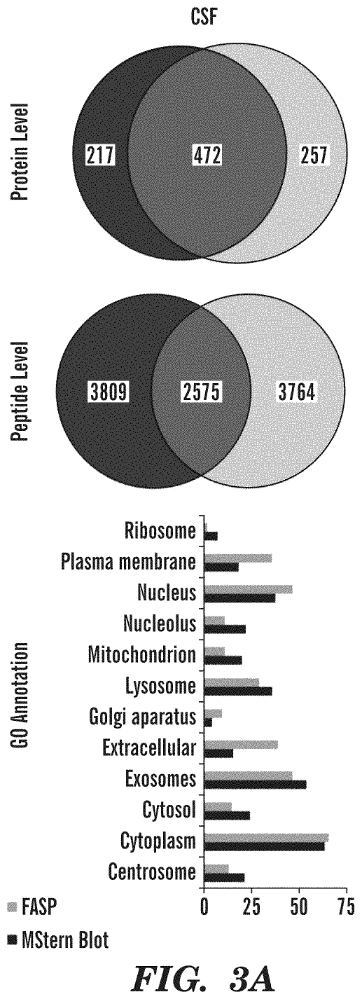

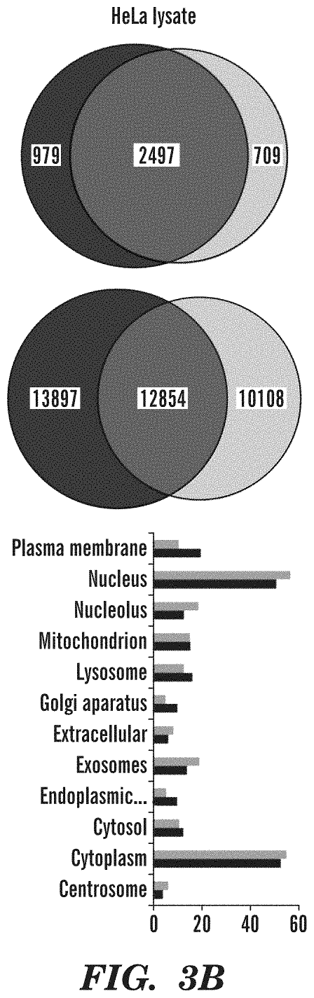

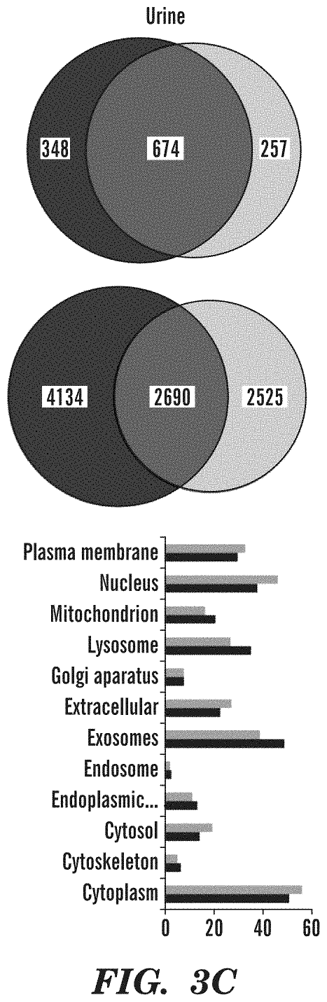

Since MStern blotting and FASP have very different modes of retention, both methods might exhibit different preferences for protein identification. The identification overlap from the combined search results of the four MStern blot and four FASP preparations of neat urine, HeLa lysate and neat CSF was compared, which were used to generate FIG. 2A. The Venn diagrams clearly show that ⅔ to ¾ of the identified proteins were shared between the MStern blot and the FASP method, while ¼ to ⅓ of the proteins are unique to either MStern blotting or FASP (FIGS. 3A-3C). The commonalities and differences at the peptide level were also compared. Here, specific peptides were in the 50 to 60% range such that only down to 40% of the observed peptides were in common.

For the subsequent GO annotation of the method-specific proteins, the funrich.org tool was used, which uses more broadly defined ontologies to make comparisons more generalizable. FIGS. 3A-3C show the results of these comparative protein localizations, whereby only the 12 most populated GO terms are listed. For neat urine and HeLa extracts, only minor differences are observable for the major GO terms. Slightly bigger differences are observable for the neat CSF, such as MStern blotting biased against plasma membrane and extracellular proteins, and a preference in favor of nucleolar, mitochondrial and/or cytosolic proteins.

Physical/Chemical Properties

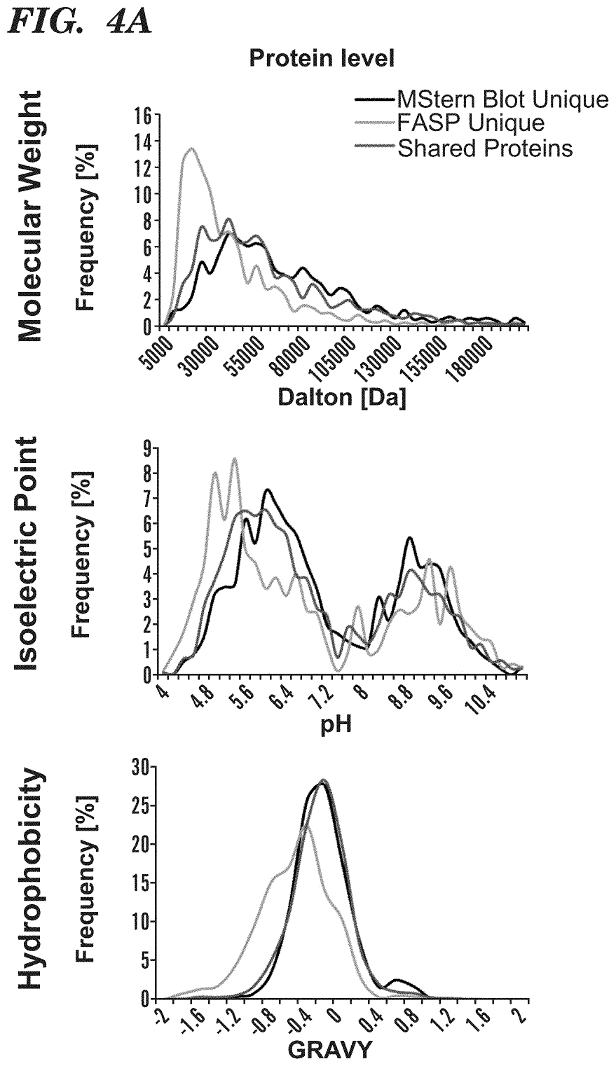

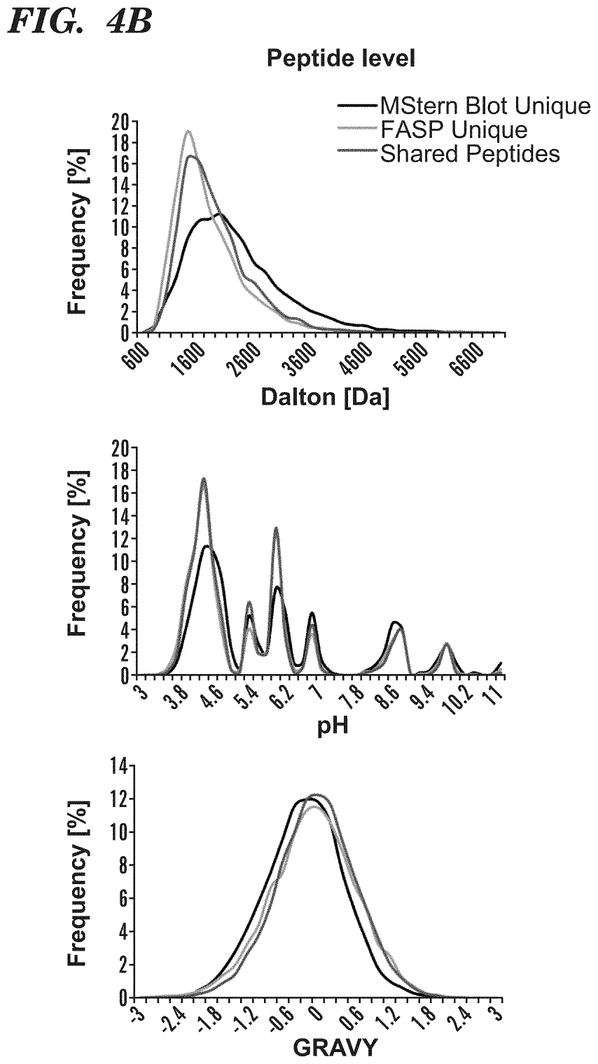

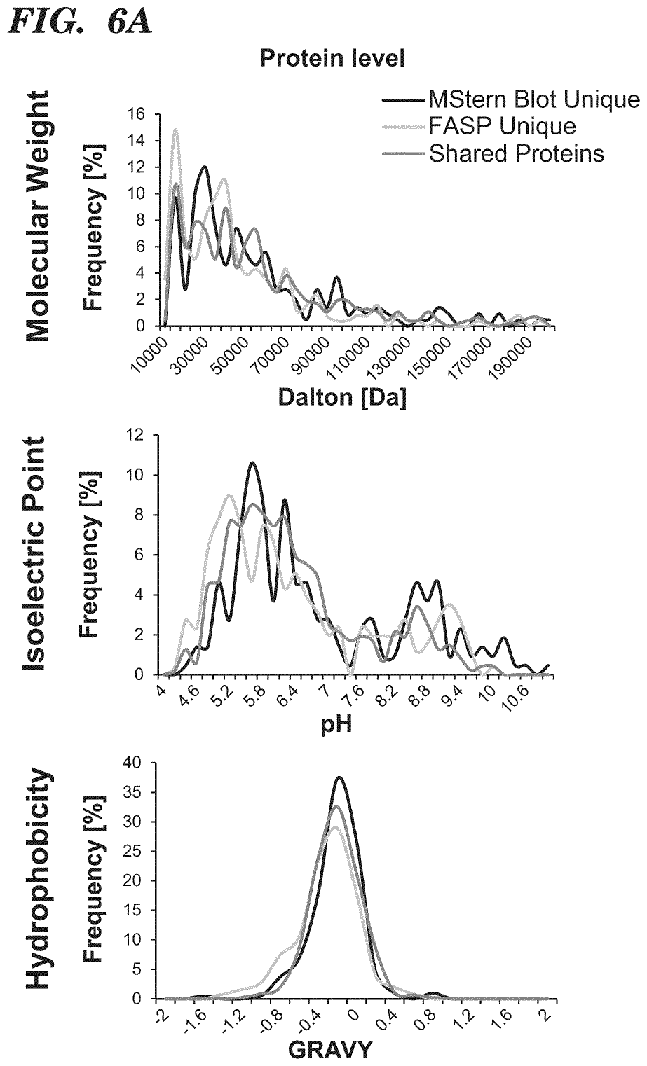

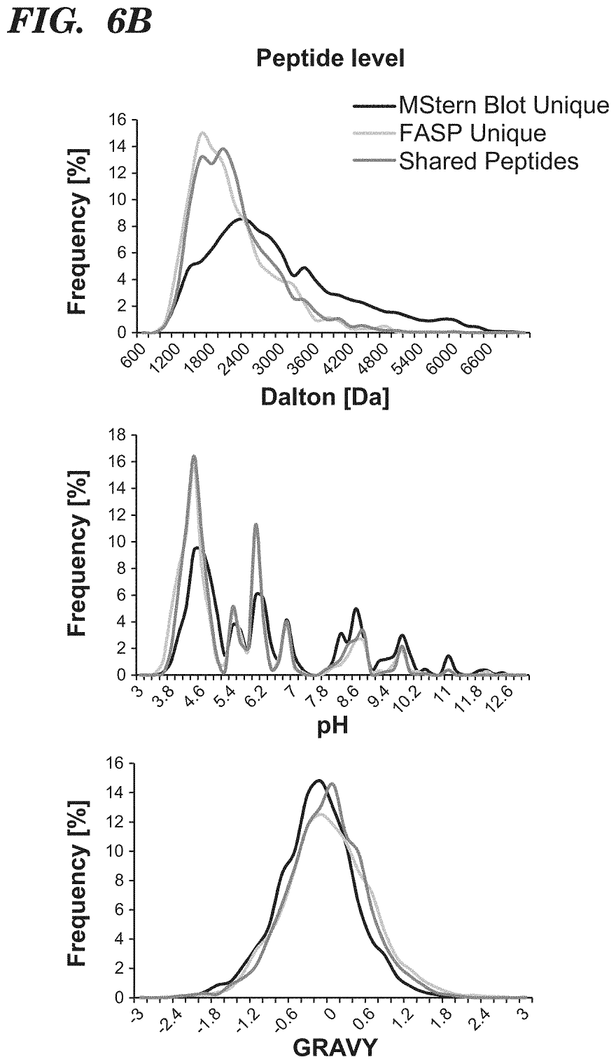

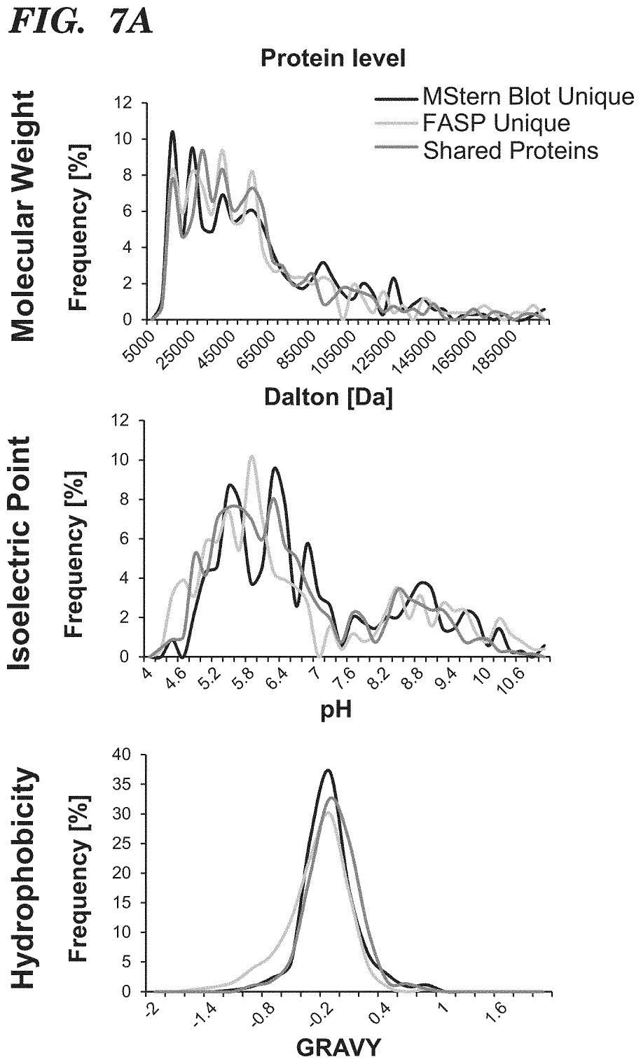

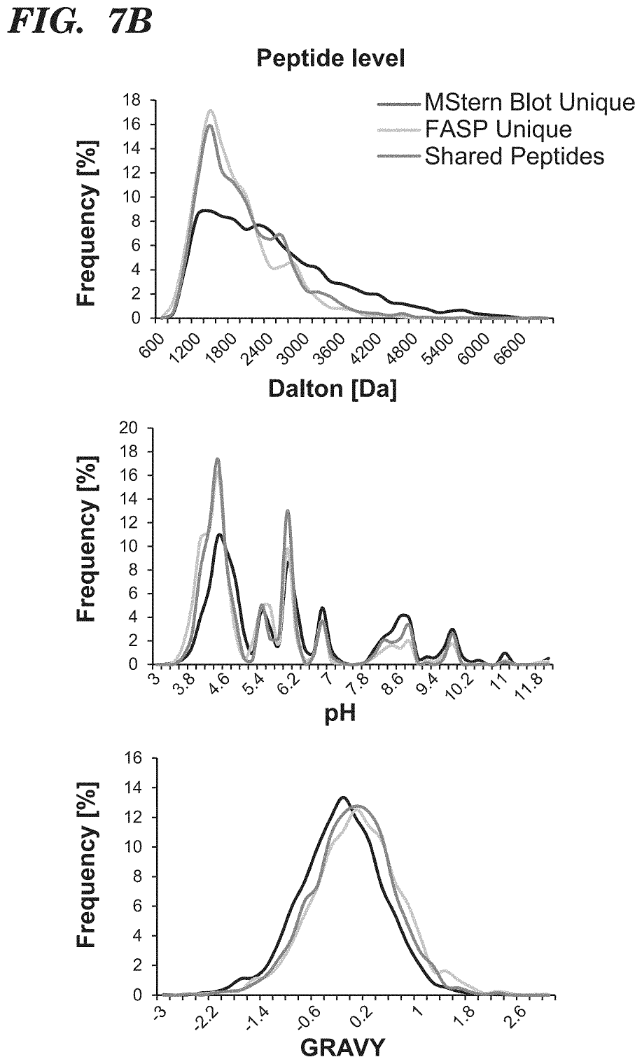

To better understand the process-specific differences in the identified proteins and peptides, the physicochemical properties of the unique and shared proteins and peptides were further probed (FIGS. 4A-4B—the graphs for the HeLa lysates are shown; the graphs for neat urine and CSF can be found in FIGS. 6A-6B, and FIGS. 7A-7B). In particular, the molecular weight, the pI and the hydrophobicity/GRAVY score were compared. Comparing the plots for the proteins (left panels), it is apparent that FASP is biasing in favor of small (low molecular weight), charged (higher and lower pI) and more hydrophilic (lower GRAVY score) proteins. In contrast, MStern blot has a slight preference for larger and less charged proteins. These observed dissimilarities match the differences in the binding modes used for the two sample processing strategies.

Comparing the physicochemical properties of the peptides (right panels) identified a major shift of the molecular weight of the MStern blot specific peptides. The MStern blot specific peptides also showed a shift away from lower pI-values in favor of higher pI values above a pI of 6.8, and a minor shift towards less hydrophilic peptides. The latter was unexpected as larger peptides are generally assumed to more hydrophobic.

Investigating the major shift in the molecular weight distributions of the observed process specific peptides revealed an increase in peptides with missed cleavages from 12.5% to 37.4% for the MStern blot vs. FASP. Attempts to modulate the degree of missed cleavages by varying the content of organic solvent[15] and/or the digestion time had only minor effects, which might indicate that the adsorption of the proteins can interfere with the trypsinization.

Protein Quantification

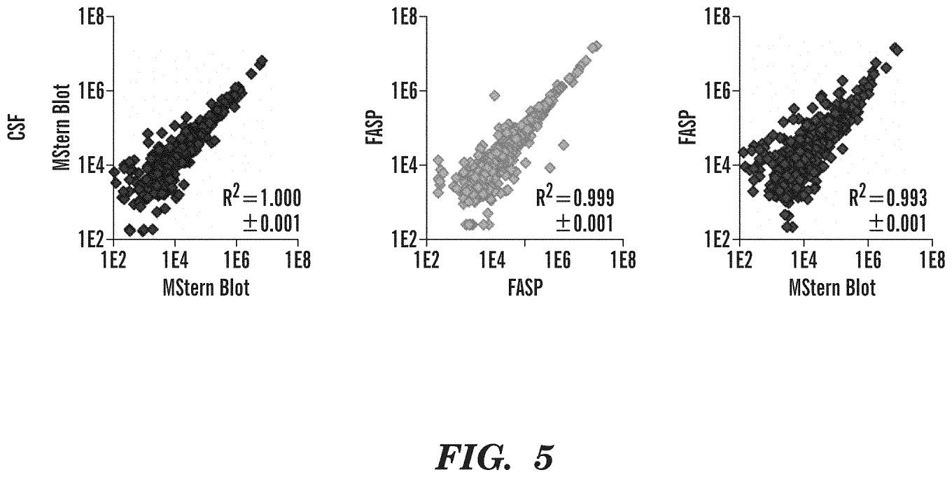

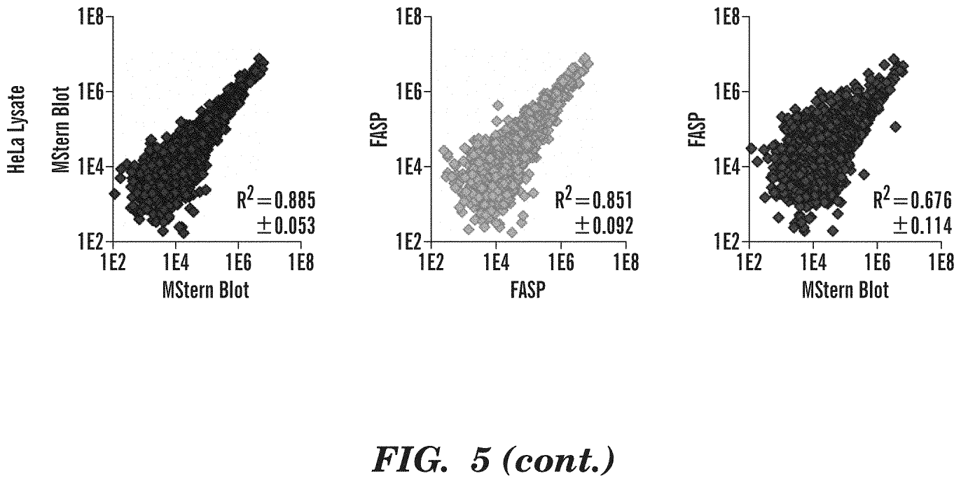

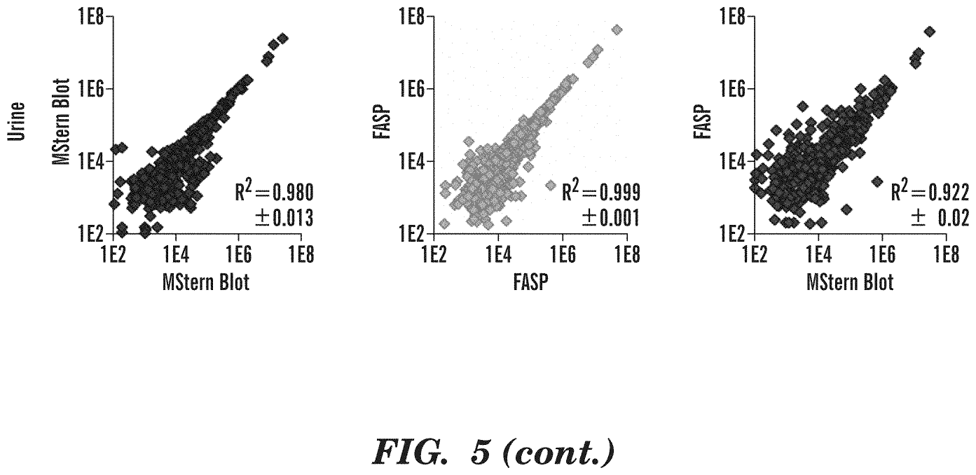

Since this degree of missed cleavages will affect the quantification of individual peptides that are not fully cleaved, the effect on the quantification of proteins was investigated. This normally uses the combined information from numerous peptides. To this end, two technical repeats of the HeLa lysates, neat urine and neat CSF digested using the MStern blotting and the FASP process were further probed (FIG. 5). Next, the peptide ion signal intensity for each protein was extracted, prior to correlating the intensities for MStern blotting vs. MStern blotting (blue), for FASP vs. FASP (yellow) and for FASP vs. MStern blotting (green). The correlations for MStern vs. MStern and FASP vs. FASP were very tight with R²-values ranging from 0.85 to 1.0. The lower correlation for the HeLa lysate had to be expected given the complex nature of the samples; this increased complexity is associated with massive undersampling, highlighting the negative effect of the stochastic nature of unbiased data dependent acquisition routines on protein quantification, which is particular limiting in the case of low abundant proteins. However, this limitation is independent of the sample processing, but can probably be improved when using e.g. non-stochastic data independent acquisition routines.

The correlation of MStern vs. FASP showed a slightly broadened scatter with R²-values ranging from 0.92 to 0.99. Based on the undersampling effect of the HeLa lysate, this sample type is considered an outlier demonstrating an R²-value of 0.67. Such slight reduction in correlation is expected when comparing two independent sample processing methods; nevertheless, the good to excellent correlations of the MStern vs. FASP-based quantification clearly shows that the increase in missed cleavage sides as observed for MStern blot-based processing still provides solid quantitative information comparable to and compatible with FASP-based processing.

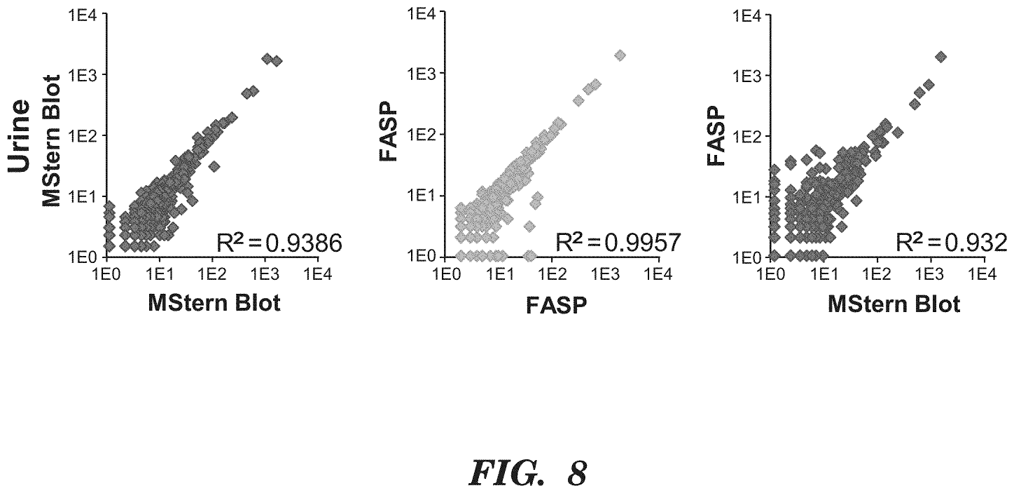

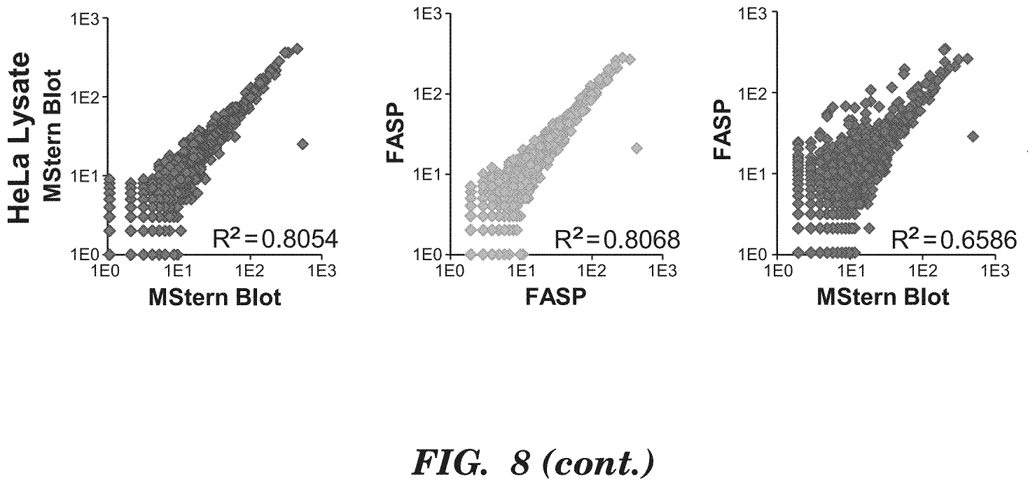

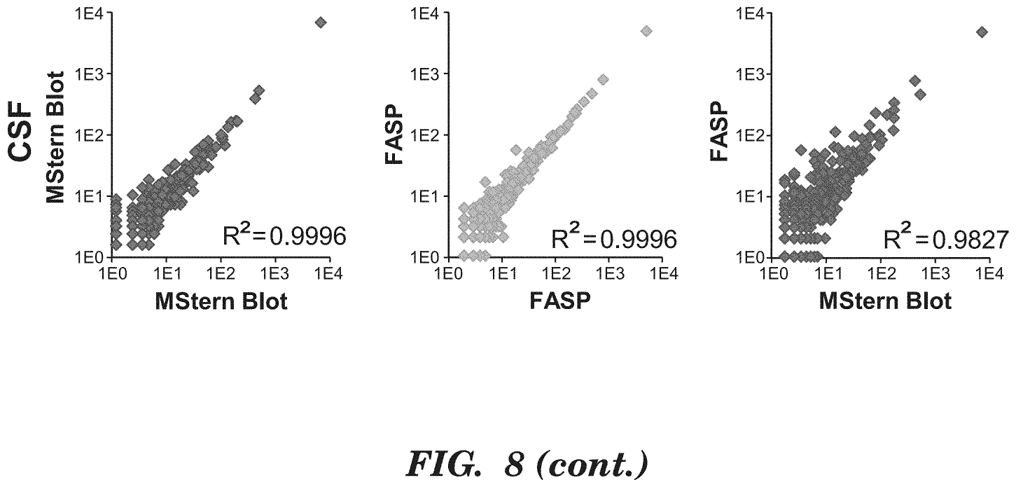

A similar analysis was performed for spectral counting-based quantitative information. The results are almost identical to the peak intensity-based quantification (FIG. 8), underscoring the notion that MStern blotting provides quantitative information of similar quality as FASP-based processing.

### CONCLUSION

Exploiting the high protein binding capacity of hydrophobic PVDF, which is also commercially available in the form of 96-well filtration plates, a 96-well plate-based sample processing method was devised, which allows for the complete processing of multiples of 96 samples or multiples thereof in a workday or less. The major time advantages compared to e.g. FASP-based protocols are the fast liquid transfers and the omission of the need for desalting digests prior to loading onto an LC/MS system. The former is the result of the 100 times larger pores when compared to ultrafiltration membranes with appropriate molecular weight cut-offs. The latter was facilitated by the efficient elution with organic solvents instead of high salt concentrations. This accelerated sample processing allows generating LC/MS-ready peptide samples, starting from ˜150 μl of neat urine, i.e. ˜15 μg of protein, in a workday or less. Although only 5 to 15 μs of protein can be processed in a single well, this amount is easily sufficient for modern LC/MS systems, onto which less than 1 μg is normally injected for each run.

The direct comparison with FASP-based processing shows that the MStern blot processing results in at least as many proteins as FASP, with an overlap of identified proteins in the 65 to 75% range, although both methods show some process-specific biases. Although MStern blot results in an increase in missed cleaved peptides, which will alter the quantification of peptides affected by the missed cleavages, it clearly shows that the quantification of proteins, which is a composite value based on numerous peptides, is not affected by this increase in missed cleaved peptides. Another major advantage of the MStern blot method is the easy compatibility with liquid handling systems, as liquid transfer is achieved using a vacuum manifold instead of a centrifuge which is necessary for, e.g., FASP-based or other sample processing protocols [16].

In summary, MStern blotting is a useful method to process dilute samples such as neat urine for downstream proteomic analysis, which lends itself to easy automation. Even though application to dilute samples such as urine is particularly advantageous, MStern is applicable to a wide range of samples without sacrificing analytical depth or quantitative nature of the data.

### REFERENCES FOR EXAMPLE 1

1. Manza, L. L., et al., Sample preparation and digestion for proteomic analyses using spin filters. Proteomics, 2005. 5(7): p. 1742-5.
2. Liebler, D. C. and A. J. Ham, Spin filter-based sample preparation for shotgun proteomics. Nat Methods, 2009. 6(11): p. 785; author reply 785-6.
3. Wisniewski, J. R., et al., Universal sample preparation method for proteome analysis. Nat Methods, 2009. 6(5): p. 359-62.
4. Switzar, L., et al., A high-throughput sample preparation method for cellular proteomics using 96-well filter plates. Proteomics, 2013. 13(20): p. 2980-3.
5. Yu, Y., et al., Urine sample preparation in 96-well filter plates for quantitative clinical proteomics. Anal Chem, 2014. 86(11): p. 5470-7.
6. Naldrett, M. J., et al., Concentration and desalting of peptide and protein samples with a newly developed C18 membrane in a microspin column format. J Biomol Tech, 2005. 16(4): p. 423-8.
7. Bradford, M. M., A rapid and sensitive method for the quantitation of microgram quantities of protein utilizing the principle of protein-dye binding. Anal Biochem, 1976. 72: p. 248-54.
8. Liu, H., R. G. Sadygov, and J. R. Yates, 3rd, A model for random sampling and estimation of relative protein abundance in shotgun proteomics. Anal Chem, 2004. 76(14): p. 4193-201.
9. Schwanhausser, B., et al., Global quantification of mammalian gene expression control. Nature, 2011. 473(7347): p. 337-42.
10. Cox, J. and M. Mann, MaxQuant enables high peptide identification rates, individualized p.p.b.-range mass accuracies and proteome-wide protein quantification. Nat Biotechnol, 2008. 26(12): p. 1367-72.
11. Eckerskorn, C. and F. Lottspeich, Structural characterization of blotting membranes and the influence of membrane parameters for electroblotting and subsequent amino acid sequence analysis of proteins. Electrophoresis, 1993. 14(9): p. 831-8.
12. Gooley, P. R., et al., The NMR solution structure and characterization of pH dependent chemical shifts of the beta-elicitin, cryptogein. J Biomol NMR, 1998. 12(4): p. 523-34.
13. Sloane, A. J., et al., High throughput peptide mass fingerprinting and protein macroarray analysis using chemical printing strategies. Mol Cell Proteomics, 2002. 1(7): p. 490-9.
14. McKeon, T. A. and M. L. Lyman, Calcium ion improves electrophoretic transfer of calmodulin and other small proteins. Anal Biochem, 1991. 193(1): p. 125-30.
15. Dickhut, C., et al., Impact of digestion conditions on phosphoproteomics8. J Proteome Res, 2014. 13(6): p. 2761-70.
16. Kulak, N. A., et al., Minimal, encapsulated proteomic-sample processing applied to copy-number estimation in eukaryotic cells. Nat Methods, 2014. 11(3): p. 319-24.

What is claimed is:

1. A method of separating a biological compound from an aqueous sample containing the biological compound, the method comprising:
(i) introducing the aqueous sample to a well of a plate, wherein the well has a bottom comprising a porous hydrophobic membrane, and the sample is in contact with a first side of the porous hydrophobic membrane;
(ii) applying a vacuum to a second side of the porous hydrophobic membrane, thereby drawing the aqueous sample through the porous hydrophobic membrane, wherein the biological compound associates with the porous hydrophobic membrane as aqueous solvent passes through; and
(iii) introducing a solvent solution to the first side of the porous hydrophobic membrane to elute the biological compound from the porous hydrophobic membrane.

2. The method of claim 1, wherein the biological compound is a peptide or polypeptide.

3. The method of claim 1, further comprising moving the hydrophobic membrane to a separate container after step (ii).

4. The method of any of claims 1-3, further comprising, after step (ii), a step of introducing a solution comprising a proteolytic enzyme to the first side of the porous hydrophobic membrane, thereby permitting the biological compound to be digested by the enzyme.

5. The method of claim 4, wherein the proteolytic enzyme is trypsin.

6. The method of claim 4, wherein the solution introduced after step (ii) comprises an organic solvent.

7. The method of claim 6, wherein the organic solvent is acetonitrile, trifluoroethanol, a combination thereof.

8. The method of any of claims 1-7, wherein the solvent solution introduced in step (iii) comprises an organic solvent.

9. The method of claim 8, wherein the organic solvent is acetonitrile, trifluoroethanol, a combination thereof.

10. The method of any one of claims 1-9, in which the average pore size of pores in the porous hydrophobic membrane is at least 50 nm in diameter.

11. The method of any one of claims 1-10, in which the average pore size of pores in the porous hydrophobic membrane is in the range of 50 nm to 5 μm in diameter.

12. The method of any one of claims 1-11, in which the average pore size of pores in the porous hydrophobic membrane is about 450 nm in diameter.

13. The method of any one of claims 1-12, wherein the porous hydrophobic membrane is comprised of a hydrophobic polymer.

14. The method of claim 13, wherein the hydrophobic polymer is selected from the group consisting of polyvinylidene difluoride (PVDF), polytetrafluoroethylene (PTFE), polyethylene, polysulfone, and polycarbonate.

15. The method of any one of claims 1-13, wherein the solvent in the solvent solution introduced in step (iii) is selected from the group consisting of acetonitrile, formic acid, methanol, ethanol, isopropanol, and combinations thereof.

16. The method of any one of claims 1-15, further comprising, after step (ii) repeating steps (i) and (ii) on the aqueous sample having passed through the porous hydrophobic membrane.

17. The method of any one of claims 1-16, further comprising washing the porous hydrophobic membrane prior to step (iii).

18. The method of claim 8 or claim 9, wherein elution step (iii) comprises stepwise introduction and removal of solvent solution containing increasing concentrations of organic solvent.

19. The method of claim 18, wherein the elution step (iii) comprises stepwise introduction and removal of a solvent solution comprising 5%, 10%, 20% and 40% acetonitrile.

20. The method claim 18, wherein the elution step (iii) comprises stepwise introduction and removal of a solvent solution comprising 10%, 20% and 40% acetonitrile.

21. The method of any one of claims 1-20, wherein the plate comprises a plurality of wells, and wherein the bottom of each well comprises a porous hydrophobic membrane.

22. The method of claim 21, wherein the aqueous sample is introduced to the plurality of wells.

23. The method of claim 21 or claim 22, wherein the plate is a 96-well plate.

24. The method of any one of claims 1-23, wherein the aqueous sample is selected from the group consisting of a cell lysate, a tissue lysate, and a biofluid.

25. The method of claim 24, wherein the biofluid is selected from the group consisting of urine, blood, serum, cerebrospinal fluid, and plasma.

26. The method of any one of claims 1-25, wherein the aqueous sample is drawn through the porous hydrophobic membrane at a flow rate in the range of 50 uL/min to 1000 uL/min.
